(19) United States
(12) Patent Application Publication
(10) Pub. No.: US 2021/0110931 A1
(43) Pub. Date: Apr. 15, 2021

(54) SYSTEMS AND METHODS OF MEDICAL PROGNOSTIC MODELING FOR TREATMENT OF TRAUMATIC BRAIN INJURIES AND OTHER MEDICAL CONDITIONS

(71) Applicant: Duke University, Durham, NC (US)

(72) Inventors: Cyrus Elahi, Durham, NC (US); Thiago Rocha, Durham, NC (US); Joao Ricardo Nickenig Vissoci, Durham, NC (US)

(21) Appl. No.: 16/833,792

(22) Filed: Mar. 30, 2020

Related U.S. Application Data
(60) Provisional application No. 62/827,259, filed on Apr. 1, 2019.

(51) Int. Cl. G16H 50/30; G16H 20/40; A61B 5/00

(52) U.S. Cl. CPC G16H 50/30; G16H 20/40; A61B 5/7475; A61B 5/4064; A61B 5/4836

(57) ABSTRACT

Systems and methods of medical prognostic modeling for treatment of traumatic brain injuries and other medical conditions are disclosed. According to an aspect, a method may use a medical treatment manager for providing a medical prognostic model for treatment of a predetermined medical condition. Further, the method includes receiving data of health of a subject. The method also includes receiving data of a treatment environment of the subject. Further, the method includes determining a treatment plan for the subject based on the medical prognostic model, the data of health of the subject, and the data of the treatment environment of the subject.

### CROSS REFERENCE TO RELATED APPLICATION

This application claims priority to U.S. Provisional Patent Application No. 62/827,259, filed Apr. 1, 2019, and titled TRAUMATIC BRAIN INJURY DECISION SUPPORT TOOL AND METHODS OF USING SAME, the content of which is incorporated herein by reference in its entirety.

### TECHNICAL FIELD

The presently disclosed subject matter relates generally to healthcare and medical technologies. Particularly, the presently disclosed subject matter relates to systems and methods of medical prognostic modeling for treatment of traumatic brain injuries and other medical conditions.

### BACKGROUND

Annually, over 10 million deaths and hospitalization are related to traumatic brain injury (TBI), with a preponderance of cases occurring in low- and middle-income countries (LMICs). Many TBI patients require surgery to prevent permanent disability or death. Unfortunately, the global neurosurgery capacity cannot meet the demand. Healthcare providers in these countries are forced to ration limited resources often without the support of computed tomography (CT) scanners or other diagnostic technologies and equipment. Therefore, there is a need for technologies to assist healthcare providers in such areas to treat patients with TBI and other conditions.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 is a block diagram of a system of medical prognostic modeling for treatment of traumatic brain injuries and other medical conditions in accordance with embodiments of the present disclosure;

FIG. 2 is a flow diagram of a method for medical prognostic modeling for treatment of a medical condition in accordance with embodiments of the present disclosure;

FIG. 3 is a block diagram of another system of medical prognostic modeling for treatment of traumatic brain injuries and other medical conditions in accordance with embodiments of the present disclosure;

FIG. 4 is a flow diagram of a method for medical prognostic modeling for treatment of a medical condition in accordance with embodiments of the present disclosure;

FIG. 5 illustrates a flow diagram depicting how the traumatic brain injury decision support tool as provided herein is used in examples scenarios in accordance with embodiments of the present disclosure; and

FIGS. 6A and 6B depict a computer's display and a smartphone, respectively, that are displaying patient risk of a poor outcome with no surgery and patient risk of a poor outcome with surgery in accordance with embodiments of the present disclosure.

### SUMMARY

The presently disclosed subject matter relates to systems and methods of medical prognostic modeling for treatment of traumatic brain injuries and other medical conditions. According to an aspect, a method may use a medical treatment manager for providing a medical prognostic model for treatment of a predetermined medical condition. Further, the method includes receiving data of health of a subject. The method also includes receiving data of a treatment environment of the subject. Further, the method includes determining a treatment plan for the subject based on the medical prognostic model, the data of health of the subject, and the data of the treatment environment of the subject.

According to another aspect, a method includes using a medical treatment manager for receiving medical treatment outcome data and associated data of health of a plurality of subjects. Further, the method includes generating and maintaining a medical prognostic model of a predetermined medical condition based on the medical treatment outcome data and associated data of health of the plurality of subjects. The method also includes determining one of a treatment plan and a likelihood of the predetermined medical condition for another subject based on data of health of the other subject and application of the medical prognostic model.

### DETAILED DESCRIPTION

The following detailed description is made with reference to the figures. Exemplary embodiments are described to illustrate the disclosure, not to limit its scope, which is defined by the claims. Those of ordinary skill in the art will recognize a number of equivalent variations in the description that follows.

Articles “a” and “an” are used herein to refer to one or to more than one (i.e. at least one) of the grammatical object of the article. By way of example, “an element” means at least one element and can include more than one element.

“About” is used to provide flexibility to a numerical endpoint by providing that a given value may be “slightly above” or “slightly below” the endpoint without affecting the desired result.

The use herein of the terms “including,” “comprising,” or “having,” and variations thereof is meant to encompass the elements listed thereafter and equivalents thereof as well as additional elements. Embodiments recited as “including,” “comprising,” or “having” certain elements are also contemplated as “consisting essentially of” and “consisting” of those certain elements.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a range is stated as between 1%-50%, it is intended that values such as between 2%-40%, 10%-30%, or 1%-3%, etc. are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

The functional units described in this specification have been labeled as computing devices. A computing device may be implemented in programmable hardware devices such as processors, digital signal processors, central processing units, field programmable gate arrays, programmable array logic, programmable logic devices, cloud processing systems, or the like. The computing devices may also be implemented in software for execution by various types of processors. An identified device may include executable code and may, for instance, comprise one or more physical or logical blocks of computer instructions, which may, for instance, be organized as an object, procedure, function, or other construct. Nevertheless, the executable of an identified device need not be physically located together but may comprise disparate instructions stored in different locations which, when joined logically together, comprise the computing device and achieve the stated purpose of the computing device. In another example, a computing device may be a server or other computer located within a retail environment and communicatively connected to other computing devices (e.g., POS equipment or computers) for managing accounting, purchase transactions, and other processes within the retail environment. In another example, a computing device may be a mobile computing device such as, for example, but not limited to, a smart phone, a cell phone, a pager, a personal digital assistant (PDA), a mobile computer with a smart phone client, or the like. In another example, a computing device may be any type of wearable computer, such as a computer with a head-mounted display (HMD), or a smart watch or some other wearable smart device. Some of the computer sensing may be part of the fabric of the clothes the user is wearing. A computing device can also include any type of conventional computer, for example, a laptop computer or a tablet computer. A typical mobile computing device is a wireless data access-enabled device (e.g., an iPHONE® smart phone, a BLACKBERRY® smart phone, a NEXUS ONE™ smart phone, an iPAD® device, smart watch, or the like) that is capable of sending and receiving data in a wireless manner using protocols like the Internet Protocol, or IP, and the wireless application protocol, or WAP. This allows users to access information via wireless devices, such as smart watches, smart phones, mobile phones, pagers, two-way radios, communicators, and the like. Wireless data access is supported by many wireless networks, including, but not limited to, Bluetooth, Near Field Communication, CDPD, CDMA, GSM, PDC, PHS, TDMA, FLEX, ReFLEX, iDEN, TETRA, DECT, DataTAC, Mobitex, EDGE and other 2G, 3G, 4G, 5G, and LTE technologies, and it operates with many handheld device operating systems, such as PalmOS, EPOC, Windows CE, FLEXOS, OS/9, JavaOS, iOS and Android. Typically, these devices use graphical displays and can access the Internet (or other communications network) on so-called mini- or micro-browsers, which are web browsers with small file sizes that can accommodate the reduced memory constraints of wireless networks. In a representative embodiment, the mobile device is a cellular telephone or smart phone or smart watch that operates over GPRS (General Packet Radio Services), which is a data technology for GSM networks or operates over Near Field Communication e.g. Bluetooth. In addition to a conventional voice communication, a given mobile device can communicate with another such device via many different types of message transfer techniques, including Bluetooth, Near Field Communication, SMS (short message service), enhanced SMS (EMS), multi-media message (MMS), email WAP, paging, or other known or later-developed wireless data formats. Although many of the examples provided herein are implemented on smart phones, the examples may similarly be implemented on any suitable computing device, such as a computer.

An executable code of a computing device may be a single instruction, or many instructions, and may even be distributed over several different code segments, among different applications, and across several memory devices. Similarly, operational data may be identified and illustrated herein within the computing device, and may be embodied in any suitable form and organized within any suitable type of data structure. The operational data may be collected as a single data set, or may be distributed over different locations including over different storage devices, and may exist, at least partially, as electronic signals on a system or network.

The described features, structures, or characteristics may be combined in any suitable manner in one or more embodiments. In the following description, numerous specific details are provided, to provide a thorough understanding of embodiments of the disclosed subject matter. One skilled in the relevant art will recognize, however, that the disclosed subject matter can be practiced without one or more of the specific details, or with other methods, components, materials, etc. In other instances, well-known structures, materials, or operations are not shown or described in detail to avoid obscuring aspects of the disclosed subject matter.

As used herein, the term “memory” is generally a storage device of a computing device. Examples include, but are not limited to, read-only memory (ROM) and random access memory (RAM).

The device or system for performing one or more operations on a memory of a computing device may be a software, hardware, firmware, or combination of these. The device or the system is further intended to include or otherwise cover all software or computer programs capable of performing the various heretofore-disclosed determinations, calculations, or the like for the disclosed purposes. For example, exemplary embodiments are intended to cover all software or computer programs capable of enabling processors to implement the disclosed processes. Exemplary embodiments are also intended to cover any and all currently known, related art or later developed non-transitory recording or storage mediums (such as a CD-ROM, DVD-ROM, hard drive, RAM, ROM, floppy disc, magnetic tape cassette, etc.) that record or store such software or computer programs. Exemplary embodiments are further intended to cover such software, computer programs, systems and/or processes provided through any other currently known, related art, or later developed medium (such as transitory mediums, carrier waves, etc.), usable for implementing the exemplary operations disclosed below.

In accordance with the exemplary embodiments, the disclosed computer programs can be executed in many exemplary ways, such as an application that is resident in the memory of a device or as a hosted application that is being executed on a server and communicating with the device application or browser via a number of standard protocols, such as TCP/IP, HTTP, XML, SOAP, REST, JSON and other sufficient protocols. The disclosed computer programs can be written in exemplary programming languages that execute from memory on the device or from a hosted server, such as BASIC, COBOL, C, C++, Java, Pascal, or scripting languages such as JavaScript, Python, Ruby, PHP, Perl, or other suitable programming languages.

As referred to herein, the terms “computing device” and “entities” should be broadly construed and should be understood to be interchangeable. They may include any type of computing device, for example, a server, a desktop computer, a laptop computer, a smart phone, a cell phone, a pager, a personal digital assistant (PDA, e.g., with GPRS NIC), a mobile computer with a smartphone client, or the like.

As referred to herein, a “user interface” is generally a system by which users interact with a computing device. A user interface can include an input for allowing users to manipulate a computing device, and can include an output for allowing the system to present information and/or data, indicate the effects of the user's manipulation, etc. An example of a user interface on a computing device (e.g., a mobile device) includes a graphical user interface (GUI) that allows users to interact with programs in more ways than typing. A GUI typically can offer display objects, and visual indicators, as opposed to text-based interfaces, typed command labels or text navigation to represent information and actions available to a user. For example, an interface can be a display window or display object, which is selectable by a user of a mobile device for interaction. A user interface can include an input for allowing users to manipulate a computing device, and can include an output for allowing the computing device to present information and/or data, indicate the effects of the user's manipulation, etc. An example of a user interface on a computing device includes a graphical user interface (GUI) that allows users to interact with programs or applications in more ways than typing. A GUI typically can offer display objects, and visual indicators, as opposed to text-based interfaces, typed command labels or text navigation to represent information and actions available to a user. For example, a user interface can be a display window or display object, which is selectable by a user of a computing device for interaction. The display object can be displayed on a display screen of a computing device and can be selected by and interacted with by a user using the user interface. In an example, the display of the computing device can be a touch screen, which can display the display icon. The user can depress the area of the display screen where the display icon is displayed for selecting the display icon. In another example, the user can use any other suitable user interface of a computing device, such as a keypad, to select the display icon or display object. For example, the user can use a track ball or arrow keys for moving a cursor to highlight and select the display object.

The display object can be displayed on a display screen of a mobile device and can be selected by and interacted with by a user using the interface. In an example, the display of the mobile device can be a touch screen, which can display the display icon. The user can depress the area of the display screen at which the display icon is displayed for selecting the display icon. In another example, the user can use any other suitable interface of a mobile device, such as a keypad, to select the display icon or display object. For example, the user can use a track ball or times program instructions thereon for causing a processor to carry out aspects of the present disclosure.

As referred to herein, a “computer network” may be any group of computing systems, devices, or equipment that are linked together. Examples include, but are not limited to, local area networks (LANs) and wide area networks (WANs). A network may be categorized based on its design model, topology, or architecture. In an example, a network may be characterized as having a hierarchical internetworking model, which divides the network into three layers: access layer, distribution layer, and core layer. The access layer focuses on connecting client nodes, such as workstations to the network. The distribution layer manages routing, filtering, and quality-of-server (QoS) policies. The core layer can provide high-speed, highly-redundant forwarding services to move packets between distribution layer devices in different regions of the network. The core layer typically includes multiple routers and switches.

As used herein, “treatment,” “therapy” and/or “therapy regimen” refer to the clinical intervention made in response to physiological condition (e.g., traumatic brain injury (TBI) manifested by a patient or to which a patient may be susceptible. The aim of treatment/therapy/therapy regimen includes the alleviation or prevention of symptoms, and/or slowing or stopping the progression or worsening of the condition.

As used herein, the term “healthcare centers” refers to any center which provides healthcare to a patient/subject. Examples include, but are not limited to, hospitals, outpatient clinics/treatment centers, doctor offices, skilled nursing facilities, field hospitals, and the like.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the patient comprises a human. In certain embodiments, the subject/patient is one who is at risk of suffering from, or suffering from, a traumatic brain injury.

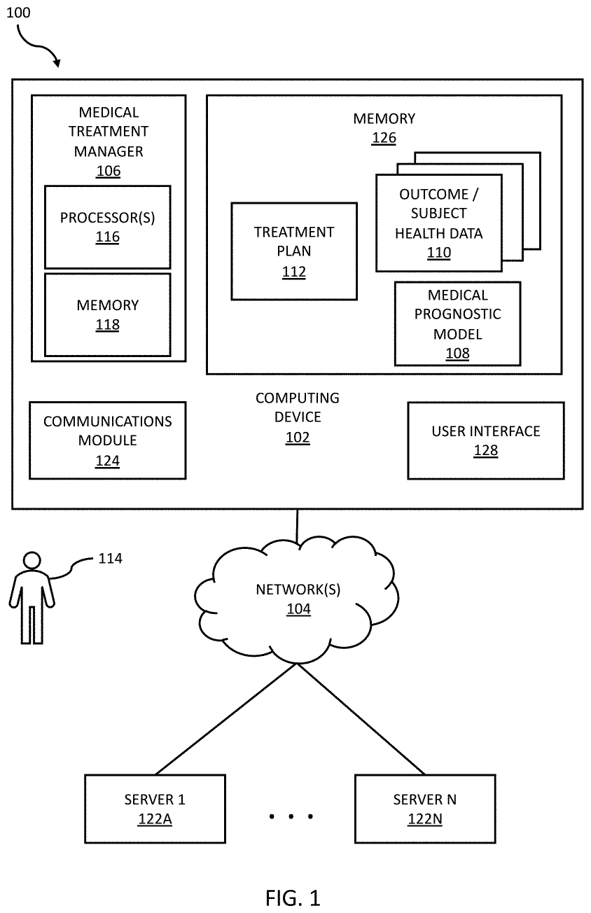

FIG. 1 illustrates a block diagram of a system 100 of medical prognostic modeling for treatment of traumatic brain injuries and other medical conditions in accordance with embodiments of the present disclosure. Referring to FIG. 1, the system 100 includes a computing device 102 that is communicatively connected to one or more other computing devices via one or more networks 104. The computing device 102 includes a medical treatment manager 106 configured to provide a medical prognostic model 108, to receive data 110 of health of subject, to receive data of treatment environments of the subjects. Further, the medical treatment manager 106 can determine a treatment plan 112 for a particular subject 114 (e.g., a patient) based on the medical prognostic model 108, the data of health of the subject 114, and the data of the treatment environment of the subject 114. The medical treatment manager 106 can also present the treatment plan 112 via a user interface (e.g., a display). The medical treatment manager 106 may include hardware, software, firmware, or combinations thereof for implementing the functionality described herein. For example, the medical treatment manager 106 may include one or more processors 116 and memory 118 for implementing the functionality described herein.

With continuing reference to FIG. 1, the system 100 includes multiple servers 1-N, which indicates multiple servers and designated as 122A-122N. Servers 122A-122N may reside at or otherwise may be associated with medical facilities, such as hospitals and medical clinics. For example, a computing device at a medical facility may be used by a medical practitioner to input various patient data. Example patient data includes, but is not limited to, a midline shift measure, a size of head bleed, a measure of pressure in a subject's brain (intracranial pressure), optic nerve sheath diameter, pupillometry, vital signs (e.g., blood pressure, pulse oximetry, heart rate, etc.), and pupils' reaction to light, demographic information, injury details, and time from injury to onset of care. The patient data may be entered or otherwise collected by a computing device at the medical facility and subsequently communicated to a respective server (e.g., one of servers 122A-122N). Further, medical treatment outcome data related to the patient data may be entered and communicated to the computing device 102. The patient data and medical treatment outcome data may be communicated from the servers 122A-122N to computing device 102 where the patient data and medical treatment outcome data can be used to generate analysis datasets. The computing device may include a communications module 124 for communicating with the network(s) 104. The medical treatment manager 106 may use the analysis datasets to build one or more medical prognostic models for treatment of one or more predetermined medical conditions. Example medical conditions include, but are not limited to, traumatic brain injury or another injury linked to traumatic brain injury, such as intracranial hemorrhage, intracranial aneurysms, arteriovenous malformations, and cavernous malformations. As an example, the medical treatment manager 106 may build the medical prognostic model 108 for treatment of a traumatic brain injury or another injury linked to traumatic brain injury.

In accordance with embodiments, a medical prognostic model may be used for predicting risk of a subject having a poor outcome from a traumatic injury and/or for determining a treatment plan for the subject. More generally, a medical prognostic model in accordance with embodiments disclosed herein may be used for predicting likelihood of one or more outcomes from a medical condition and/or for determining a treatment plan for the subject. In some embodiments, systems and methods disclosed herein can use an algorithm that utilizes statistical techniques to generate individual patient risk estimates and locally-relevant prognostic models that can calculate a patient risk for a bad outcome in a particular geographic location, hospital, other treatment facility. Further, systems and methods disclosed herein can optimize resource allocation for the mid- to low-resource setting, thereby allowing, for example, hospitals, health systems, treatment facilities, insurance companies, and government health offices in low- and middle-income countries to improve quality of care and reducing health care costs.

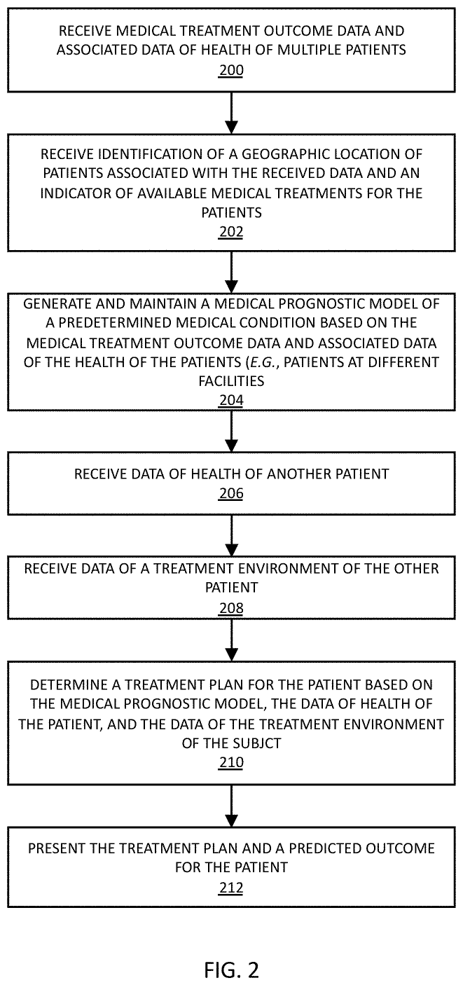

FIG. 2 illustrates a flow diagram of a method for medical prognostic modeling for treatment of a medical condition in accordance with embodiments of the present disclosure. The method may be used, for example, for treatment of traumatic brain injury. It is noted that the method is described by example as being implemented by the system 100 shown in FIG. 1, although the method may alternatively be implemented by any other system.

Referring to FIG. 2, the method includes receiving 200 medical treatment outcome data and associated data of health of multiple subjects. For example, the servers 122A-122N may receive patient data includes, but is not limited to, a midline shift measure, a size of head bleed, a measure of pressure in a patient's brain, a vital sign, pupil reaction to light, and the like. A medical practitioner, such as a physician, nurse, or other healthcare worker, may enter the data directly at one of the servers or another computing device that may be connected to the server. Each server 122A-122N may communicate their received data to the computing device 102 where the data can be stored as record data 110 in memory 126. Further, information about the patient outcome associated with the data of the health of the patient may be received at servers 122A-122N and subsequently communicated to the computing device 102 where the data can be stored as record data 110 in memory 126. The medical treatment manager 106 may receive and store the received data in memory 126.

The method of FIG. 2 includes receiving 202 identification of a geographic location of patients associated with the received data and an indicator of available medical treatment for the patients. Continuing the aforementioned example, the servers 122A and 122N may communicate to the computing device 102 an identification of their geographic location and/or an indicator of available medical treatment for patients at the associated healthcare facility.

The method of FIG. 2 includes generating and maintaining 204 a medical prognostic model of a predetermined medical condition based on the medical treatment outcome data, associated data of the health of the patients, data from patients with the same condition previously treated at that facility, as well as data from patients at other facilities with the same condition. Continuing the aforementioned example, the medical treatment manager 106 may generate and maintain the medical prognostic model 108 based on the received data 110. The medical prognostic model 108 can be a model for treatment of traumatic brain injury or another predetermined medical condition. The medical prognostic model 108 can be represented by an equation with variables that may be set by one or more of a patient's midline shift measure, a size of head bleed, a measure of pressure in the patient's brain, and the like for treatment of a medical condition of the patient. The medical prognostic model 108 may also be based upon the identified geographic location and/or an indicator of available medical treatment for patients. For example, an equation representing the model 108 may have variables including identified geographic location and/or an indicator of available medical treatment.

The medical treatment manager 106 can provide the model 108 for use by healthcare practitioners for treatment of a predetermined medical condition of a particular patient. For example, the patient 114 may arrive at a healthcare facility with traumatic brain injury or an injury linked to traumatic brain injury (e.g., a head injury in an automobile accident). A healthcare practitioner attending the patient 114 may enter available data of the health of the patient (e.g., vital signs, blood pressure, etc.) into the computing device 102 for use by the manager 106 for application to the model 108 to determine the treatment plan 112 for the patient and predicting a treatment outcome for the patient.

For example, with continuing reference to FIG. 2, the method includes receiving 206 data of health of another patient. Continuing the aforementioned example, the patient 114 may arrive at a hospital with a head injury. At the time of arrival, an attending healthcare practitioner may be uncertain as to whether the head injury resulted in traumatic brain injury for the patient 114. The healthcare practitioner may collect data of the health of the patient, such as vital signs (e.g., pulse oximetry, temperature, respiration rate, heart rate, systolic blood pressure, diastolic blood pressure, and/or the like). The healthcare practitioner may use a user interface 128 of the computing device 102 to enter the collected data for use by the manager 106 for recommending treatment of the patient 114 and/or predicting an outcome for the patient 114. Alternatively, for example, the data may be collected elsewhere at a different computing device and subsequently communicated to the computing device 102 for use by the manager 106 for recommending treatment of the patient 114 and/or predicting an outcome for the patient 114.

The method of FIG. 2 includes receiving 208 data of a treatment environment of the other patient. Continuing the aforementioned example, the medical treatment manager 106 may receive data of a treatment environment of the other patient. For example, the data of the treatment environment can indicate a geographic location of the patient 114, an availability of healthcare equipment or qualified healthcare practitioners for treating the patient for traumatic brain injury. For example, an indicator of available medical treatment for a subject may include, but is not limited to, an indicator of number of available operating rooms, number of ICU beds, availability of rehabilitation services after discharge, availability of intracranial pressure (ICP) monitoring, the like, and combinations thereof. This data may be stored in memory 126.

The method of FIG. 2 includes determining 210 a treatment plan for the patient based on the medical prognostic model, the data of health of the patient, and the data of the treatment environment of the patient. Continuing the aforementioned example, the medical treatment manager 106 can determine the treatment plan 112 for the patient 114 based on the medical prognostic model 108, the data of health of the subject, data from similar patients at other facilities, and the data of the treatment environment of the subject. The medical treatment manager 106 can also determine a predicted outcome for the patient by use of the model 108.

The method of FIG. 2 includes presenting 212 the treatment plan and a predicted outcome for the patient. Continuing the aforementioned example, the medical treatment manager 106 can present the treatment plan and the predicted outcome for the patient 112 via the user interface 128. In this way, a healthcare practitioners can be provided a recommended treatment plan and predicted outcome for a patient given a set of circumstances for the patient, e.g., the patient's health data and the treatment environment of the patient.

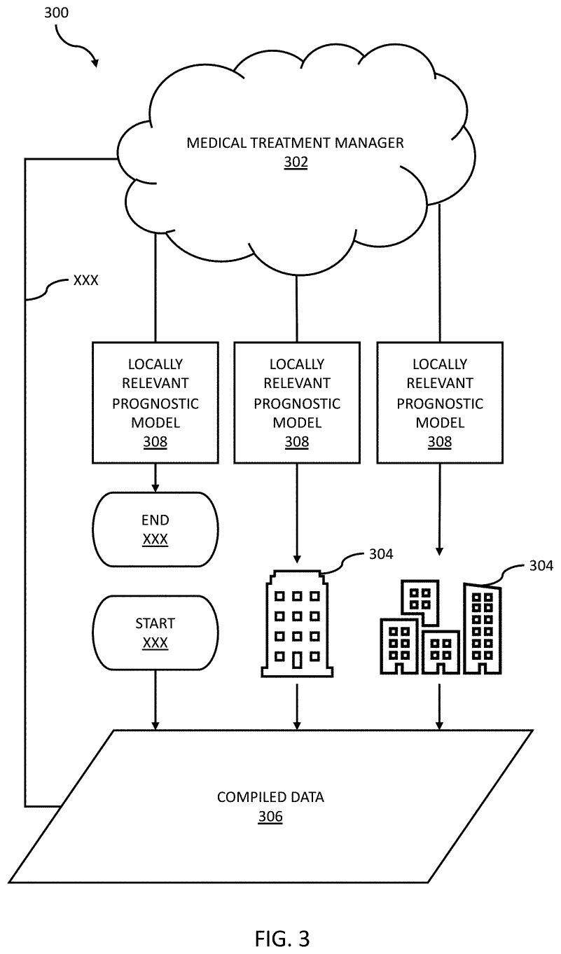

FIG. 3 illustrates a block diagram of another system 300 of medical prognostic modeling for treatment of traumatic brain injuries and other medical conditions in accordance with embodiments of the present disclosure. Referring to FIG. 3, the system 300 depicts a computer-implemented environment where an end-user (e.g., a medical professional, health care provider, etc.) can interact with a medical treatment manager 302 in accordance with embodiments of the present disclosure. The medical treatment manager 302 provides, in this example, a traumatic brain injury decision support tool. It should be understood that this system 300 may be similarly applied as a decision support tool for treating another type of health condition. The medical treatment manager 302 may be implemented in one or more computing devices (e.g., the computing device 102 shown in FIG. 1), but it is represented here as residing in the “cloud,” one or more servers connected via the Internet and/or other networks.

In some embodiments, the medical treatment manager 302 can assist healthcare practitioners at medical facilities 304 (e.g., a hospital) to input de-identified patient data, represented as being moved to storage as compiled data 306. The compiled data 306 may be suitably stored and provided to the medical treatment manager 302. For example, the compiled data 306 may be stored in one or more servers. The medical treatment manager 302 may use the compiled data from all hospitals using this platform 306 for creating analysis datasets and for building one or more tailored medical prognostic models to the individual hospital 308 for identifying/determining whether a subject may suffer a poor outcome from a traumatic brain injury. In some embodiments, the data 306 includes, for example vital signs and physical exam findings which can be obtained by providers with limited resources and only basic training. Such data may include, but is not limited to, the data shown in Table 1 below.

TABLE 1
Patient Variables

| | | | |
|---|---|---|---|
| Age | Alcohol Use | Pupils (e.g., | Intention of |
| | (y/n) | bilateral | Injury |
| | | reactive, | (e.g., |
| | | unilateral | unintentional, |
| | | reactive, | self-inflicted, |
| | | nonreactive, | inflicted by |
| | | unknown) | other, |
| | | | unknown) |
| Vital Signs (e.g., | Disposition from | Gender | Pulse |
| Pulse Oximetry, | the Emergency | | |
| Temperature, | Department (e.g., | | |
| Respiration Rate, | ICU, Surgery, | | |
| Heart Rate, | Operating Theater, | | |
| Systolic | Home, Death) | | |
| blood pressure, | | | |
| Diastolic blood | | | |
| pressure) | | | |
| PERRLA (Pupils | Surgical Ward to | Clinical Exam: | TBI Surgery |
| equal round | ICU (y/n/unknown) | GCS (Glasgow | (y/n/unknown) |
| reactive to | | Coma Scale) | |
| light) | | | |
| (y/n) | | | |
| Other Surgery | | | |
| (y/n/unknown) | | | |

In other embodiments, the medical treatment manager 302 can implement the prognostic model(s) 308 to predict the risk of a subject having a traumatic brain injury and if so, the manager 302 can provide a suggested or recommended treatment regimen for the subject. In some embodiments, the medical treatment manger 302 can combine machine learning based on the prognostic model(s) 308 and based on various patent variables. In embodiments and as shown in FIG. 3, the machine learning can combine the target hospital data with data from other hospitals sharing similar contexts of care to generate robust, locally-relevant prognostic model 308. In some embodiments, the medical treatment manger 302 can build the prognostic models 308 using one or more deterministic models/algorithms. For example, the medical treatment manger 302 can implement one or more machine learning algorithms, such as Bayesian Generalized Linear Model algorithm, Ridge Regression algorithm, Gradient Boosting Machine algorithm, Bayesian Additive Regression Trees algorithm, Bagged Tree algorithm, Neural network algorithm, K nearest neighbor algorithm, penalized logistic regression algorithm, Random Forests algorithm, Support Vector Machine algorithm, C5.0, and combinations thereof, for building the one or more prognostic models. It should be understood that other known machine learning algorithms may also be implemented for building the prognostic models 308. In some embodiments, a prognostic model 308 can be returned to the target hospital using mobile application on a smartphone, tablet computer, or other computing device.

In embodiments, the medical treatment manger 302 can assist one or more of the users to build and/or evaluate prognostic models 308 through computing device having a graphical user interface, such as a smartphone, tablet computer, or laptop computer. For example, the users (or the system operator) may provide inputs at the graphical user interface of the computing device for the medical treatment manager 302 to build the prognostic model(s) 308. In some embodiments, the user inputs may include information relating to a patient's vital signs and/or physical exam findings as disclosed herein.

A user can interact with the medical treatment manager 302 in a number of ways, such as over one or more networks. For example, one or more servers accessible through the network(s) can host the medical treatment manager 302. The one or more servers can also store or have access to the one or more data stores for storing data for the TBI decision support tool, or receive input data (e.g., patient vital signs and/or physical exam results) from external sources. It should be appreciated that in alternative embodiments the server may be self-contained and not connected to external networks due to security or other concerns.

With continuing reference to FIG. 3, patient data can be obtained by and entered by a user to create the compiled data 306 that is then fed into the medical treatment manager 302 to generate one or more training datasets and/or one or more testing datasets. One or more models may subsequently be built using the one or more training datasets. Prediction result data of the one or more models based at least in part on the one or more training datasets can be given as inputs to an ensemble algorithm for generating ensemble data as a final set of predictions. The medical treatment manager 302 can determine one or more final prognostic models based at least in part on the ensemble data. The medical treatment manager 302 can apply the one or more final prognostic models 308 to the one or more test datasets for performance evaluation to generate evaluation results. The one or more final prognostic models 308 can be applied to new patient data of an individual patient for the identification/determination of the presence of a traumatic brain injury in the patient.

In some embodiments, a data handling process can be performed (e.g., by the medical treatment manager 302) to obtain the inputted data as disclosed herein. In embodiments, a stratified random approach can be used to generate the one or more training datasets and/or the one or more testing datasets with a numeric regimen categorization and response category so that regimens for affected subjects are proportionally represented in the training datasets and the testing datasets. One or more machine learning algorithms, including but not limited to, penalized logistic regression, random forests, and/or C5.0, can be applied (e.g., by the medical treatment manager 302) on the one or more training datasets for prognostic model building. In some embodiments, a penalized logistic regression algorithm is implemented to find parameter estimates that maximize the objective function (e.g., log-likelihood), subject to regularization constraints. One regularization constraint forces the parameter estimates to be smaller (e.g. shrinkage), while the other regularization constraint essentially forces some parameter estimates to zero (e.g. lasso). The penalized logistic regression algorithm is suited for problems where the predictors are highly correlated or when there are more predictors than subjects. Because the regularization forces some parameter estimates to zero, a prognostic model generated based on the penalized logistic regression algorithm performs internal variable selection.

In other embodiments, the random forests (RF) algorithm is a tree-based method built on an ensemble of trees. A prognostic model generated based on the RF algorithm does the following process many times: selects a bootstrap sample of the training dataset and builds a tree on the bootstrap sample. Within each tree, a randomly selected number of predictors can be chosen, and the optimal split can be selected only from that sample. One or more tuning parameters for prognostic model generated based on the RF algorithm include the number of randomly selected predictors for each split. Building an ensemble of trees in this way can reduce the variance seen by using just a single tree.

In yet other embodiments, the C5.0 algorithm can be used to generate a predictive model. For example, in some embodiments the C5.0 algorithm is implemented (e.g., TBI decision support tool 105) to build a sequence of trees. At each step in the sequence, the TBI decision support tool 105 adjusts each sample's weight based on the accuracy of the model at each iteration. Samples that are predicted correctly are given less weight, while samples that are predicted incorrectly are given more weight. The final model prediction is a combination of the predictions across all trees. It should be understood that the systems and methods disclosed herein are not limited to penalized logistic regression, random forests, and C5.0 that are merely described above as examples. Other machine learning algorithms may be implemented for prognostic modeling.

In some embodiments, the ensemble algorithm can be trained to combine the prediction result data optimally to generate the ensemble data. For example, a weight may be determined for the prediction result of each of the models, and a weighted sum of the prediction results of all the models is calculated to generate the ensemble data.

In some embodiments, the ensemble algorithm can involve an average calculation of the result data generated by applying the models on the training datasets. In some embodiments, the ensemble algorithm can use a logistic regression model to combine the result data across models. It should be understood that the ensemble algorithm disclosed herein is not limited to the average calculation and the logistic regression model. The ensemble algorithm may include a stacking technique, a blending technique, or any other known second-level machine learning algorithm in which predictions of a collection of models are combined to form a final set of predictions.

In some embodiments, the final predictive models are applied to the testing datasets to generate the evaluation results. As an example, the evaluation results include sensitivity or specificity parameters for the one or more final predictive models. In some embodiments, the one or more final prognostic models provided herein can be used to predict the presence of a TBI and the prognosis in a subject with an accuracy of at least 85%, 86%, 87%, 88%, 89%, 90%, 91%, 92%, 93%, 94%, 95%, 96%, 97%, 98%, or at least 99%.

In some embodiments, a system for predicting the risk of a subject having a traumatic brain injury can include a computing system that contains one or more processors, memory, and a medical treatment manager as disclosed herein. The computing system may include any suitable type of computing device (e.g., a server, a desktop, a laptop, a tablet, a mobile phone, etc.) that includes the processor or provide access to a processor via a network or as part of a cloud-based application. The TBI decision support tool may also be implemented as part of a user interface module (e.g., a mobile application).

In some embodiments, the present disclosure provides a computing system for predicting the presence of a traumatic brain injury in a subject, the computing system comprising a processor, one or more memory devices, one or more input/output devices, one or more networking components, and a system bus. In some embodiments, the computing system includes the medical treatment manager, and provides access to the medical treatment manager to a user as a stand-alone computer.

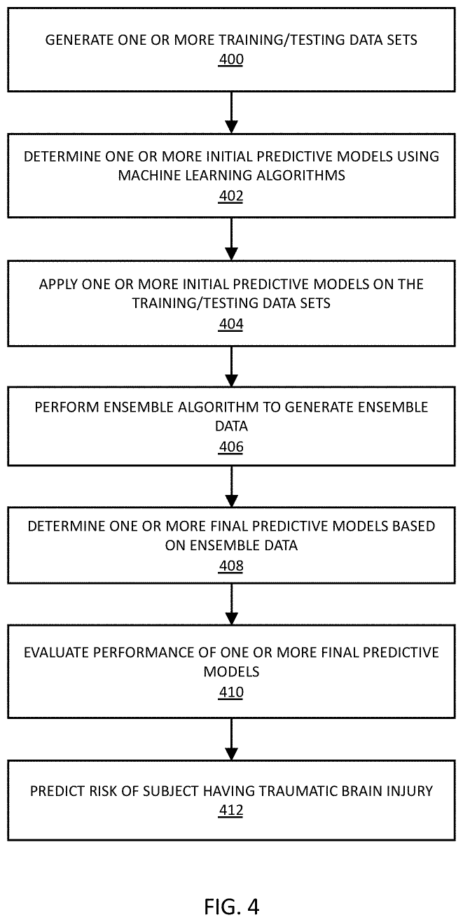

FIG. 4 illustrates a flow diagram of a method for medical prognostic modeling for treatment of a medical condition in accordance with embodiments of the present disclosure. The method may be used, for example, for determining the risk of a subject having a poor outcome (e.g., death) from a traumatic brain injury. It is noted that the method is described by example as being implemented by the system 100 shown in FIG. 1, although the method may alternatively be implemented by any other system.

Referring to FIG. 4, the method includes generating 400 one or more training datasets and/or one or more testing datasets based at least in part on patient variables from multiple patients. Subsequently, the method includes determining 402, using one or more data processors, one or more initial predictive models based on the subject's data using one or more machine learning algorithms comprising at least in part on one or more training data sets and/or one or more testing datasets based at least in part of a plurality of patient variables. The method also includes applying 404 one or more initial predictive or prognostic models to the one or more training datasets to generate result data. Further, the method includes performing 406 an ensemble algorithm on the result data to generate ensemble data. Based at least in part on the ensemble data, the method includes determining 408 one or more final predictive or prognostic models. Subsequently, the method includes evaluating 410 the performance of the one or more of the final predictive or prognostic models. The method also includes predicting 412 the risk of the subject having a traumatic brain injury. In some embodiments, the method may also include implementing an appropriate plan for treating the subject(s).

Further, the tool may be used for diagnosis and prognosis. For example, in the case of traumatic brain injury being suspected for a patient, the tool may be used for determining the patient's risk for having a poor outcome (e.g., death). As an example, the tool may determine a percentage of the risk of death of the patient (e.g., 60% or 99% chance of death). Based on provision of this risk by the tool, a healthcare practitioner can triage the patient accordingly.

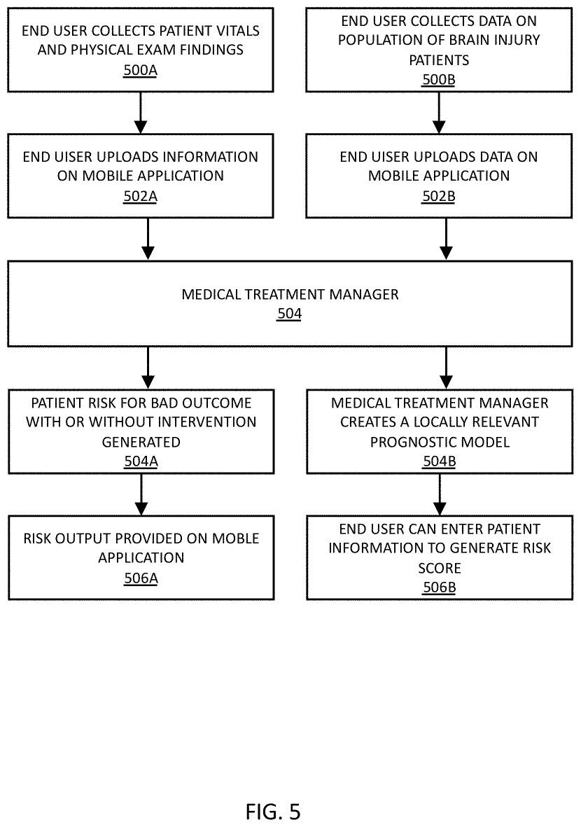

In embodiments, systems and methods disclosed herein, the medical treatment manager, such as manager 106 shown in FIG. 1, can assist a user to utilize the tool for individual patients (e.g., predicting the risk of a subject having a poor outcome from a traumatic brain injury) and/or evaluate and/or build one or more prognostic models for identifying/determining the likelihood of a subject having a traumatic brain injury or other medical condition. FIG. 5 illustrates a flow diagram depicting how the traumatic brain injury decision support tool as provided herein is used in examples scenarios in accordance with embodiments of the present disclosure. The method may be used, for example, for treatment of traumatic brain injury. It is noted that the method is described by example as being implemented by the system 100 shown in FIG. 1, although the method may alternatively be implemented by any other system.

Referring to FIG. 5 and particularly the left side (or side “A”) of the flow diagram, an end-user enters patient variables relating to the subject into a medical treatment manager 504 (blocks 302A and 302A). Once entered into the medical treatment manager, the medical treatment manager 106 can determine one or more initial prognostic models based on the subject's data using one or more machine learning algorithms comprising at least in part on one or more training data sets and/or one or more testing datasets based at least in part of multiple patient variables. These one or more initial prognostic models on the one or more training datasets can subsequently be used to generate result data, which are subsequently run against an ensemble algorithm to generate ensemble data. Based on the ensemble data, one or more final prognostic models is created and evaluated. The result of the algorithmic process results in a patient risk score indicating the probability of the patient having a poor outcome and the need medical intervention (block 504A). For example, in embodiments, the patient risk score is indicative of a “bad” outcome with or without medical intervention (e.g., 75% risk for bad outcome with surgery vs. 90% risk for bad outcome without surgery). The results can displayed on the display screen of the computing device being used by the user (e.g., smartphone, tablet, laptop, etc.) (block 506A). In some embodiments, the method can include implementing an appropriate plan for treating the subject(s), wherein the TBI decision support tool can offer recommendations for treatment to be implemented by the user.

Referring now to the right side (or side “B”) of the flow diagram of FIG. 5, the method depicts an example flow chart for building and evaluating predictive platform models to generate a locally-relevant traumatic brain injury decision support tool in accordance with embodiments of the present disclosure. At blocks 500B and 502B, data is collected by one or more end-users and uploaded into the medical treatment manager 504. Subsequently, one or more training datasets and/or one or more testing datasets based at least in part on patient variables from a plurality of patients that are provided by the one or more end-users from potentially multiple facilities can be generated. Further, one or more initial prognostic models can be determined using one or more machine learning algorithms based at least in part on the one or more training data sets as previously described. For example, the one or more machine learning algorithms correspond to one or more of the following: penalized logistic regression, random forests, and C5.0. The one or more initial prognostic models can be applied on the one or more training data sets to generate result data. Subsequently, an ensemble algorithm can be performed on the result data to generate ensemble data. For example, the ensemble algorithm may correspond to an average calculation or a logistic regression model. At block 504B, one or more final prognostic models can be determined based at least in part on the ensemble data. At block 506B, performance of the one or more final prognostic models can subsequently be evaluated based at least in part on the one or more test datasets, where an end-user can then enter patient information into the TBI decision support tool to receive a risk assessment based on one or more of the final prognostic models.

In embodiments, data of health of a patient may include any available and suitable intensive magnetic resonance imaging (MRI), computed tomography (CT) image data, and/or any other data collected by medical equipment. This collected data may be combined with other data disclosed herein for use by the medical treatment manager for generating a medical prognostic model. Further, the model may be used by the medical treatment manager for determining a treatment plan for a subject and/or determining a patient's risk as disclosed herein.

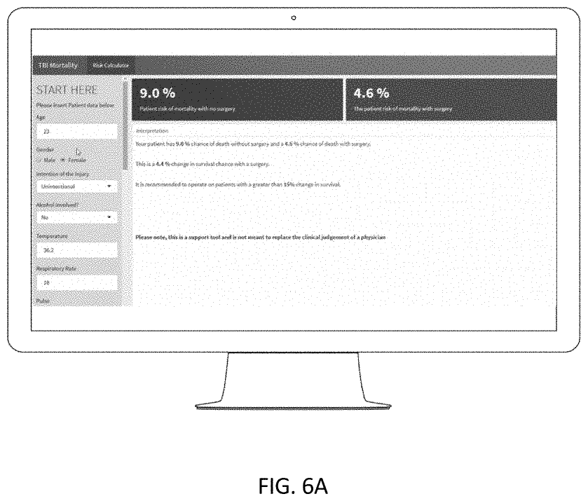

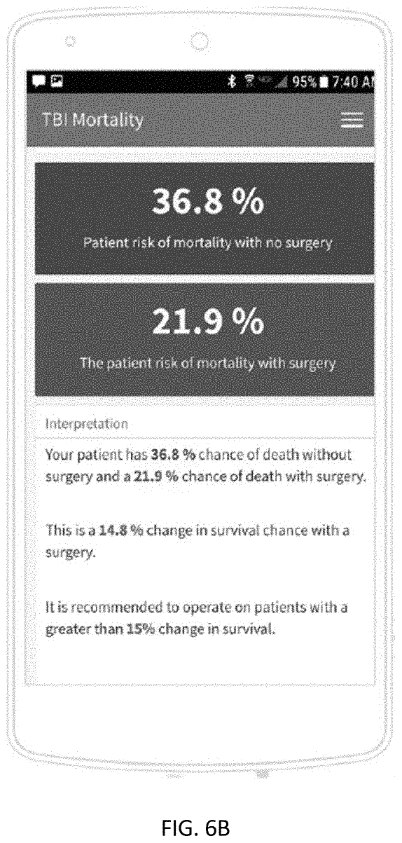

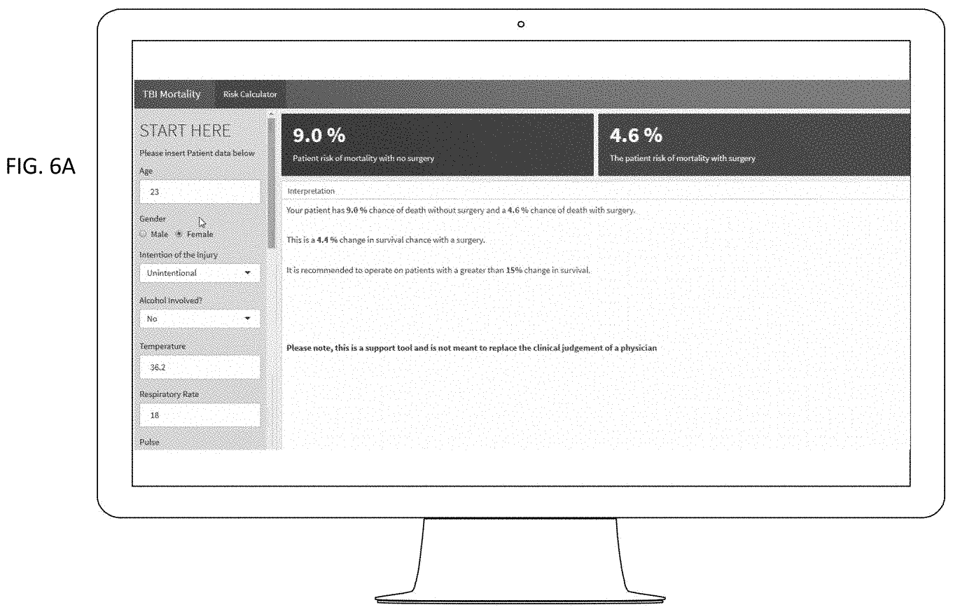

FIGS. 6A and 6B illustrate a computer's display and a smartphone, respectively, that are displaying patient risk of a poor outcome with no surgery and patient risk of a poor outcome with surgery in accordance with embodiments of the present disclosure. Referring to FIG. 6A, in this example, the patient risk of mortality with no surgery is 9.0%, and the patient risk of mortality with surgery is 4.6%. Referring to FIG. 6B, in this example, the patient risk of mortality with no surgery is 36.8%, and the patient risk of mortality with surgery is 21.9%. These percentages may be determined by a medical treatment manager in accordance with embodiments of the present disclosure.

In accordance with embodiments, the endpoint for a prognostic model is the Glasgow outcome scale extended (GOSe) score. The GOSe score can be provided at patient discharge. The discharging physician or a research nurse can calculate the GOSe score on all patients at hospital discharge unless patient mortality occurred during the hospitalization. The GOSe is an ordered outcome from one (worst outcome) to eight (best outcome), one represents patient death while eight represents upper good recovery. The GOSe scores were dichotomized into good and poor recovery. Scores were classified from one to six as poor recovery and scores of seven or eight as good recovery. A dichotomous outcome was chosen rather than ordinal because in a clinical setting, there may be few patients with moderate outcomes. Scores were selected less than seven, rather than less than five, as poor recovery to increase the number of patient with poor recovery. This step can improve the balance between good and poor recovery in the dataset. However, with more balanced dataset the dichotomization can occur at different thresholds (e.g., poor outcome=GOSe of 1-4; good outcome=GOSe of 5-8).

In an example application in accordance with embodiments of the present disclosure, all data were processed using the statistical language R. The first step included handling missing data. The outcome variable had no missing data. Input variables with more than 80% of cases missing were removed. The only variable removed during this first analysis was the glucose level. Next, multiple imputation were used using chained equations to impute missing values from variables with less than 20% of cases missing. Each variable was separated considering the measurement level (e.g., numeric, categorical) for different imputation approaches. All the variables for the imputation process were considered. The resulting dataset was obtained after 10 iterations.

A high correlation analysis was performed following the missing imputation steps to identify high correlation variables (i.e., greater than 0.9). No variable was dropped during this step. An analysis to exclude outliers among the numeric variables was performed and the following cases were dropped: age greater than 75 years, respiratory rate greater than 75, and systolic blood pressure greater than 220 mmHg.

Several variables considered in the analysis were ordinal or categorical. As a result, the initial group of 21 variables expanded to 56 after dummy variable conversion. To maintain data integrity after this step, the data was investigated for the presence of near zero variance, high correlation and linear combos. Eighteen variables were removed after the near zero variance test, seven were removed after the analysis of high correlation, and the linear combo analysis did not exclude any variables. Ultimately, data from 3138 patients in the database and 31 variables were used after all preprocessing steps.

A patient based cross validation with 10 folds and five repetitions was used for data splitting. Since the outcome of interest was imbalanced (14.4% had a poor recovery), a regularization was used for imbalanced procedure called Synthetic Minority Over-sampling Technique (SMOTE).

Nine different models were tested: k-Nearest Neighbor, Ridge Regression, Neural Network, Bagged Tree, Bayesian Generalized Linear Model, Bayesian Additive Regression Trees, Gradient Boosting Machine, Single C5.0 Ruleset and Random Forest. All methods were validated using an internal approach. The kappa statistic was the metric used to assess the prognostic models built. The metrics for comparison among the models were based on confusion matrix statistics: area under the ROC curve (AUC), sensitivity, specificity, positive predictive value, negative predictive value. All AUC comparative analysis were performed using the R, pROC package

In one population of patients admitted to a healthcare facility, 2685 (85.6%) had a good recovery while 453 (14.4%) patients had a poor recovery. There were 321 (71%) mortality cases in the poor recovery group. Both the good and poor recovery groups were predominantly male (82%, 83%) with unintentional injuries (81%, 87%). The mean age was also similar, 30.7 and 33.9 years for the good and poor recovery groups respectively. Seven-hundred and two (26.2%) of those in the good recovery group and 112 (24.7%) in the poor recovery group reported use of alcohol at time of injury.

The vital signs among good and poor recovery groups were not significantly different. However, this was not the case for clinical examination variables. The average GCS score of the poor recovery group was 8.7 while the good recovery group had a mean score of 14. In the good recovery group, 2641 (98.4%) patients had bilaterally reactive pupils compared to 318 (70.2%) in the poor recovery group. In the good recovery group, 554 (21%) patients were transferred from the surgical ward to the ICU compared to 242 (53%) in the poor recovery group. One-hundred-and-forty-nine (33%) patients in the poor recovery group received TBI surgery compared with 551 (21%) in the good recovery group.

Nine different ML models were tested on the dataset. The AUC of the models varied from 66.2% (K nearest neighbors) to 86.5% (Bayesian Generalized Linear Model). Despite this range, several models obtained similar AUC values. A confidence interval analysis of AUC identified the Bayesian Generalized Linear Model as the best approach to categorize the outcomes. For traumatic brain injury patients receiving care at the medical facility, this model produced a CI 95%: 85.6-87.4 and an AUC of 86.5.

The Bayesian generalized linear model had a sensitivity of 0.89 and a specificity of 0.67. The precision was 0.94. The accuracy of the model was of 0.86 and the kappa statistic was 0.49. The best model in this particular application achieved a moderate to high capability of predicting a good recovery and an intermediate ability of predicting a bad recovery.

The predictive weight of each variable in the Bayesian generalized linear model was extracted. The top four predictors of a good outcome were an increasing GCS score, an increasing blood oxygen level, a foot or fist mechanism of injury (MOI) and if the injury was unintentional. The top four predictors of a poor outcome were a patient being sent to the intensive care unit (ICU) after surgery, a MOI by a gun, direct admission to the surgical floor, and a car MOI.

Machine learning based prognostic models can be used to increase efficiency and precision of decision making for neurosurgical conditions including traumatic brain injury. In a survey of HIC and LMIC physicians who treat patients with head injury, accurate prognostic information was considered very important for the following: deciding to undertake a decompressive craniotomy, deciding who should receive intensive care, communication with patient families and which patients' treatment should be withdrawn. One study exploring how a prediction system influences intensity of management based on injury severity supports the potential for a more rational use of hospital resources. The researchers found more intensive management for patients with moderate or good prognosis. Conversely, for those with a worse prognosis, the frequency of using resources for intensive management decreased by 39%. These findings also support the prediction of poor outcome rather than prediction of good outcome. In resource poor settings, prediction of poor outcome may result in more prudent use of allocation of limited resources. Conversely, prediction of good outcome may increase resources dedicated to a patient, problematic in a setting devoid of resource.

An example application of a prognostic model for the hospital in this study is the decision to send a patient to the ICU (intensive management) or surgical ward (less intensive management). It was found that 53% of patients who ultimately were classified as having poor outcome at discharge had been transferred from the surgical ward to the ICU prior to discharge compared to only 21% of patients classified as having a good outcome at discharge. This finding means patients were assessed in the ED, deemed sufficiently stable for care on the surgical ward, and later required transfer to the ICU for more aggressive care. The significant difference between the good and poor recovery group requiring this transfer represents an opportunity to improve initial triaging decisions in the ED. This example is a potential application for prognostic models to support complex clinical decision making in hospital settings with a limited number of highly-skilled healthcare providers.

In an example scenario of use, an end-user enters a series of patient metrics into a medical treatment manager. The medical treatment manager can use a previously-generated algorithm derived from data similar to the end-user's context of care. Further, the medical treatment manager can produce a patient risk score for bad outcome with or without a treatment (e.g., 75% risk for bad outcome with surgery vs 90% risk for bad outcome without surgery).

In another example scenario of use, an end-user can upload a de-identified (no identifiable patient information like name) brain injury dataset from his or her health care center (target hospital) to a medical treatment manager. The medical treatment manager can use this data, along with data from other health care centers similar to the target hospital, to create a locally relevant decision support tool. The end-user can then perform an algorithm as disclosed herein on this decision support tool tailored to his or her health care center.

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the embodiments have been described in connection with the various embodiments of the various figures, it is to be understood that other similar embodiments may be used, or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed is:

1. A method comprising:
using a medical treatment manager for:
providing a medical prognostic model for treatment of a predetermined medical condition;
receiving data of health of a subject;
receiving data of a treatment environment of the subject; and
determining a treatment plan for the subject based on the medical prognostic model, the data of health of the subject, and the data of the treatment environment of the subject.

2. The method of claim 1, wherein the predetermined medical condition is traumatic brain injury or another injury linked to traumatic brain injury.

3. The method of claim 1, wherein the medical prognostic model is represented by an equation with variables comprising one or more of a midline shift measure, a size of head bleed, a measure of pressure in a subject's brain, optic nerve sheath diameter, pupillometry, vital signs, and pupils' reaction to light, optic nerve sheath diameter, demographic information, injury details, and time from injury to onset of care.

4. The method of claim 3, wherein receiving data of health of the subject comprises receiving data of the subject to apply to the variables,
wherein determining the treatment plan comprises determining the treatment plan by application of the received data of the subject to the medical prognostic model.

5. The method of claim 4, further comprising presenting the treatment plan via a user interface.

6. The method of claim 1, wherein the medical prognostic model is represented by an equation with variables comprising one or more of a geographic location of the subject and an indicator of available medical treatment for the subject.

7. The method of claim 6, wherein receiving data of health of the subject comprises receiving data of the subject to apply to the variables,
wherein determining the treatment plan comprises determining the treatment plan by application of the received data of the subject to the medical prognostic model.

8. The method of claim 7, further comprising presenting the treatment plan via a user interface.

9. The method of claim 1, further comprising:
receiving the data of health of the subject and the data of the treatment of the subject from an identified geographic location; and
sending the treatment plan for the subject to a requesting computing device, and
wherein determining the treatment plan comprises applying the identified geographic location to the medical prognostic model.

10. A system comprising:
a medical treatment manager including at least one processor and memory configured to:
provide a medical prognostic model for treatment of a predetermined medical condition;
receive data of health of a subject;
receive data of a treatment environment of the subject; and
determine a treatment plan for the subject based on the medical prognostic model, the data of health of the subject, and the data of the treatment environment of the subject.

11. The system of claim 10, wherein the medical prognostic model is represented by an equation with variables comprising one or more of a geographic location of the subject and an indicator of available medical treatment for the subject.

12. The system of claim 11, wherein receiving data of health of the subject comprises receiving data of the subject to apply to the variables,
wherein determining the treatment plan comprises determining the treatment plan by application of the received data of the subject to the medical prognostic model.

13. The system of claim 12, further comprising presenting the treatment plan via a user interface.

14. The system of claim 10, further comprising:
receiving the data of health of the subject and the data of the treatment of the subject from an identified geographic location; and
sending the treatment plan for the subject to a requesting computing device, and
wherein determining the treatment plan comprises applying the identified geographic location to the medical prognostic model.

15. A method comprising:
using a medical treatment manager for:
receiving medical treatment outcome data and associated data of health of a plurality of subjects;
generating and maintaining a medical prognostic model of a predetermined medical condition based on the medical treatment outcome data and associated data of health of the plurality of subjects; and
determining one of a treatment plan and a likelihood of the predetermined medical condition for another subject based on data of health of the other subject and application of the medical prognostic model.

16. The method of claim 15, further comprising using the medical treatment manager for determining data of a treatment environment of the plurality of subjects, and
wherein generating and maintaining the medical prognostic model comprising generating and maintaining the medical prognostic model based on the determined data of the treatment environment of the plurality of subjects.

17. The method of claim 15, wherein the predetermined medical condition is traumatic brain injury or another injury linked to traumatic brain injury.

18. The method of claim 15, wherein the medical prognostic model is represented by an equation with variables comprising one or more of a midline shift measure, a size of head bleed, a measure of pressure in a subject's brain, optic nerve sheath diameter, pupillometry, vital signs, and pupils' reaction to light, optic nerve sheath diameter, demographic information, injury details, and time from injury to onset of care.

19. The method of claim 15, wherein the medical prognostic model is represented by an equation with variables comprising one or more of a geographic location of the subject and an indicator of available medical treatment for the subject.

20. A system comprising:
a medical treatment manager including at least one processor and memory configured to:
receive medical treatment outcome data and associated data of health of a plurality of subjects;
generate and maintaining a medical prognostic model of a predetermined medical condition based on the medical treatment outcome data and associated data of health of the plurality of subjects; and
determine one of a treatment plan and a likelihood of the predetermined medical condition for another subject based on data of health of the other subject and application of the medical prognostic model.
